(19) United States
(12) Patent Application Publication
(10) Pub. No.: US 2020/0377515 A1
(43) Pub. Date: Dec. 3, 2020

(54) PROCESS FOR THE PREPARATION OF DIHALOBENZOPHENONES, NEW CHEMICALS USEFUL FOR ITS IMPLEMENTATION AND METHODS FOR PREPARING SAID CHEMICALS

(71) Applicant: RHODIA OPERATIONS, Aubervilliers (FR)

(72) Inventor: Eric MULLER, Lyon (FR)

(21) Appl. No.: 16/764,184

(22) PCT Filed: Nov. 30, 2018
(86) PCT No.: PCT/EP2018/083243

§ 371 Date: May 14, 2020

(30) Foreign Application Priority Data
Dec. 1, 2017 (EP) 17306691.1

(51) Int. Cl. C07D 493/08; C07C 45/74

(52) U.S. Cl. CPC C07D 493/08; C07C 49/813; C07C 45/74

(57) ABSTRACT

The invention relates to new compounds of formulae (IV) and (VII): (IV) (VII) wherein X represents a halogen atom selected from the group consisting of fluorine, chlorine, bromine and iodine and R is a hydrocarbyl radical, said compounds being useful as precursors and/or intermediates for the preparation of 4,4′dihalobenzophenones of formula (I): (I) wherein X is as defined above.

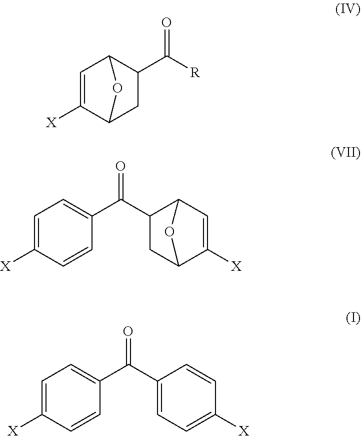

This application claims priority to European application No. 17306691.1 filed on Dec. 1, 2017, the whole content of this application being incorporated herein by reference for all purposes.

### TECHNICAL FIELD

The present invention pertains to new and useful specific chemical compounds which are obtainable by cycloaddition between either a (vinyl)(hydrocarbyl)ketone or a (vinyl)(phenyl)ketone and a halo furan (Diels-Alder reaction) and to their use as precursors and/or intermediates for the preparation of dihalobenzophenones.

### BACKGROUND ART

Dihalobenzophenones are valuable chemical compounds which are known to the art. They are mainly prepared by oxidation of the methylene group of a dihalodiphenyl methane to provide the corresponding dihalobenzophenone. Various other methods are known, such as those described in U.S. Pat. No. 4,943,358, WO 2012/001131, U.S. Pat. No. 5,777,172 and in Dunlop et al., “The preparation of 4-fluoro-and 4,4′-difluorobenzophenone”, J. Am. Chem. Soc., 1933, 55(4), pp. 1665-1666. A non-exhaustive summary of other available processes for the manufacture of 4,4′-difluorobenzophenone may also be found in U.S. Pat. No. 7,687,668.

4,4′-difluorobenzophenone (4,4′-DFBP) is the central starting material for preparing poly(aryl ether ketone)s (PAEK) which are a well-known class of engineering polymers used in various fields. These are high-performance polymers with constantly growing annual production volumes, so that the volume of 4,4′-DFBP produced worldwide annually is also included in the growth. The most important polyether ketones are the poly(ether ketone)s (PEK) and poly(ether ether ketone)s (PEEK), which feature melting points of above 330° C. and high chemicals resistance.

The present invention aims at providing a new and efficient process for the preparation of dihalobenzophenones.

Another objective of the present invention is to provide a process for the preparation of dihalobenzophenones involving renewable starting materials.

### SUMMARY OF THE INVENTION

A first object of the present invention relates to a process for the preparation of a 4,4′-dihalobenzophenone of formula (I):

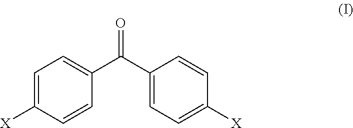

wherein X represents a halogen atom selected from the group consisting of fluorine, chlorine, bromine and iodine,

said process comprising the steps of:

a1) reacting a halo furan of formula (II) with a vinylhydrocarbylketone of formula (III):

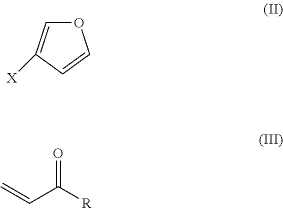

wherein X is the same as defined above, and R is a methyl radical,

thereby providing reaction products containing a cycloadduct compound of formula (IV):

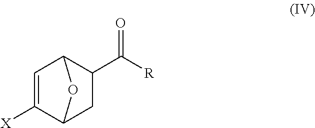

in which X and R are the same as defined above

a2) optionally further isolating said compound of formula (IV)

b) carrying out a dehydration/aromatization of said adduct compound of formula (IV), thereby providing reaction products containing a (p-halogeno)phenyl hydrocarbyl ketone of formula (V):

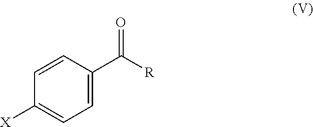

in which X and R are the same as defined above

c) carrying out an alpha-methylenation of said compound of formula (V), thereby providing reaction products containing a (p-halogeno)phenyl vinyl ketone of formula (VI):

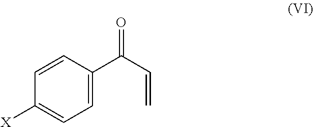

in which X is the same as defined above

d1) reacting said compound of formula (VI) with the same halofuran of formula (II) as defined above, thereby providing reaction products containing a cycloadduct compound of formula (VII):

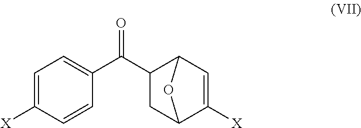

in which X is the same as defined above d2) optionally further isolating said compound of formula (VII)

e) carrying out a dehydration/aromatization of said adduct compound of formula (VII), thereby providing reaction products containing said 4,4′-dihalobenzophenone of formula (I).

The invention also relates to the compounds of formula (IV) and (VII) as defined above, which are indeed chemicals new per se.

The invention also pertains with a process for the manufacture of said compounds of formula (IV) and (VII), said process involving in both cases a Diels-Alder reaction between a halofuran and a monovinylketone.

The invention further relates to the use of a compound of formula (VII) as defined above, for the direct preparation of a 4,4′-dihalobenzophenone of formula (I), said preparation involving a dehydration/aromatization reaction step on a compound of formula (VII).

According to the invention, in all its aspects, the halogen atom X is preferably selected from bromine and chlorine, more preferably chlorine.

According to the invention, in all its aspects, the hydrocarbyl radical R may be chosen among the alkyl radicals or the aryl radicals. Preferably, R is an alkyl radical, linear or branched, substituted or unsubstituted, which may contain from 1 to 20 carbon atoms. Most preferably, R is an unsubstituted linear C1-C10 alkyl radical, even most preferably an unsubstituted linear C1-C2 alkyl radical.

According to the invention, in all its aspects, the Diels-Alder reactions may be carried out with or without the use of a solvent, and with or without the use of a catalyst.

According to an embodiment of the invention, in all its aspects, the dehydration/aromatization reactions are preferably carried out in the presence of an alkali methoxide, preferably sodium methoxide or potassium methoxide, or an alkali hydroxide, preferably sodium hydroxide or potassium hydroxide.

According to another embodiment of the present invention, the dehydration/aromatization reactions are preferably carried out in the presence of a solvent. The solvent is advantageously chosen from alcohols, sulfoxides and mixtures thereof. It is preferably chosen from C₁-C₆ (especially C₁-C₄) aliphatic alcohols, cyclohexanol, dialkyl sulfoxides wherein each out of the 2 alkyl groups contains from 1 to 6 carbon atoms (especially from 1 to 4 carbon atoms), alkyl phenyl sulfoxides wherein the alkyl group contains from 1 to 6 carbon atoms (especially from 1 to 4 carbon atoms), tetrahydrothiophene 1-oxide and mixtures thereof. Good results were obtained notably when using dimethyl sulfoxide (DMSO) as the solvent.

As already mentioned, one of the advantages of the invention is to provide a process for the preparation of 4,4′-dihalobenzophenones involving renewable starting materials.

Moreover, it has been surprisingly found that the process for the preparation of compound of formula (I) according to the invention allows satisfactory conversion and selectivity.

### DETAILED DESCRIPTION OF THE INVENTION

The rational of the different chemicals (some being new per se) and chemical steps involved in the present invention may be summarized as follows (in this scheme, R is methyl, but could also represent another hydrocarbyl radical as detailed hereinafter):

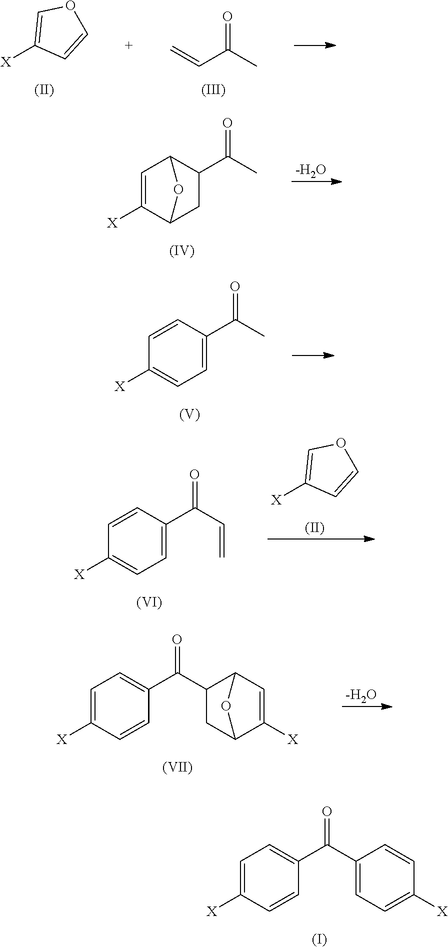

in which —H₂O symbolizes a dehydration/aromatization step carried out on a cycloadduct.

Preferably, the halo furan of formula (II) is 3-chlorofuran or 3-bromofuran.

The halofuran of formula (II) can be obtained from furan according to processes known to the person skilled in the art. Document U.S. Pat. No. 2,773,882 describes such a process.

Furan may be prepared by decarbonylation of furfural which is obtained from a renewable resource, see for example U.S. Pat. Nos. 4,764,627 and 8,754,245. Suitable renewable resources as well as suitable methods for their conversion into furfural are known to the person skilled in the art.

Alternatively, the halo furan of formula (II) may be prepared by any other conventional chemical reactions, or may be also commercially available products.

The vinylhydrocarbylketone of formula (III) can be obtained from the corresponding hydrocarbylketone and formaldehyde, for example as described by Siegel H. and Eggersdorfer M., Ullmann's Encyclopedia of Industrial Chemistry (2000). Moreover, some of these products, in particular methylvinylketone, are commercially available.

It has been surprisingly found that a Diels-Alder reaction (step a1) efficiently occurs between the halo furan of formula (II) and the vinylhydrocarbylketone of formula (III) resulting in the formation of a cycloadduct of formula (IV) as defined above. Moreover, it has been surprisingly found that said Diels-Alder reaction leads to the formation of compounds of formula (IV) with a good conversion, a low amount of by-products and a good selectivity in the desired product.

The present invention therefore also provides a process for the production of compound of formula (IV), comprising reacting a halofuran of formula (II) with a vinylhydrocarbylketone of formula (III), said two formulae being the same as defined above, and optionally further isolating compound of formula (IV).

As already mentioned, compounds of formula (IV) are new and useful products per se, and are therefore part of the present invention.

The Diels-Alder reaction between the halofuran of formula (II) and the vinylhydrocarbylketone of formula (III) leads to the formation of two main structural isomers represented below, one of which being the compound of formula (IV):

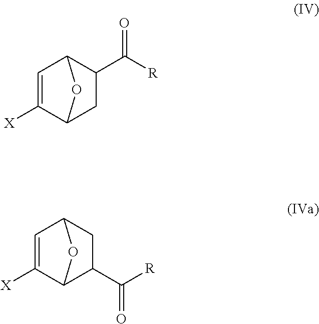

The reaction between compound of formula (II) and compound of formula (III) might also lead to the formation of byproducts in minor amounts, in particular the compound of formula (A):

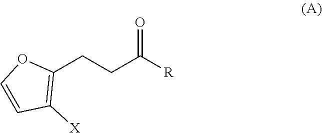

According to an embodiment of the invention, the molar ratio of compounds of formula (IV) to the compounds of formula (IVa) is of at least 50/50, preferably at least 70/30, more preferably at least 80/20, even more preferably at least 90/10.

According to an embodiment of the invention, the molar ratio of compound of formula (IV) to compound of formula (A) is of at least 50/50 or at least 60/40 or at least 80/20 or at least 90/10, and even 100/0.

According to an embodiment of the invention, the conversion of the Diels-Alder reaction is of at least 50%, preferably at least 70%, more preferably at least 90%.

The Diels-Alder reaction between the halofuran of formula (II) and the vinylhydrocarbyl ketone can be carried out under usual Diels-Alder conditions known to the person skilled in the art.

The Diels-Alder reaction may be conducted with or without a catalyst. The use of catalysts can improve kinetics and selectivity of the Diels-Alder reaction. Known Diels-Alder catalysts may be used and may include Lewis acids, such aluminium chloride, ethylaluminium dichloride, diethylaluminium chloride, trimethyl aluminium, bismuth (III) chloride, bismuth (III) trifluoromethanesulfonate, boron trifluoride, boron triacetate, cerium (III) chloride, copper (I) trifluoromethanesulfonate, copper (II) chloride, hafnium (IV) chloride, iron (II) chloride, iron (II) acetate, iron (III) chloride, iron (III) acetate, lithium perchlorate, lithium trifluoromethanesulfonate, magnesium bromide, magnesium iodide, magnesium chloride, magnesium perchlorate, scandium (III) trifluoromethanesulfonate, tin (IV) chloride, titanium (IV) chloride, titanium (IV) isopropoxide, N-trimethylsilyl-bis(trifluoromethanesulfonyl)imide, trimethylsilyl trifluoromethanesulfonate, ytterbium (III) trifluoromethanesulfonate, zinc chloride, zinc bromide, zinc iodide, zinc acetate zirconium (IV) chloride and indium (III) chloride, triphenylborane, Bronsted acids, such as inorganic mineral acids, e.g. sulphuric acid, phosphoric acid, nitric acid, hydrobromic acid or hydrochloric acid, and organic acids such as methane sulphonic acid, p-toluenesulphonic acid or carboxylic acids. One of the preferred Diels-Alder catalysts is magnesium iodide.

Alternatively, activated carbon, silica, alumina, silica-alumina, zirconia and zeolites may be used as such or as support for a catalytically active metal or metal compound. Suitable metals or metal compounds include alkali metals, alkaline earth metals, transition metals, noble metals, rare earth metals. The catalysts can be acidic, e.g. by treating supports with phosphoric acid, or by ion exchange of zeolites to render them into their acidic form. Examples of solid catalysts include amorphous silica-alumina, zeolites, preferably zeolites in their H-form, and acidic ion exchange resins. Other suitable catalysts that are liquids or that may be dissolved in the appropriate solvent to yield a homogeneous catalyst environment, include organic and inorganic acids, such as alkane carboxylic acid, arene carboxylic acid, sulphuric acid, phosphoric acid, hydrochloric acid, hydrobromic acid and nitric acid.

The Diels-Alder reaction may be carried out with or without a solvent. Suitable solvents may be selected from pyridine, tertiary amines such as triethylamine and diisopropylethylamine, chloroform, dichloromethane, diethyl ether, perfluorinated alkanes such as perfluorohexane, toluene, water and ionic liquids such as 1-butyl-3-methylimidazolium tetrafluoroborate and 1-butyl-3-methylimidazolium hexafluorophosphate, alone or in a mixture. Preferred solvents are pyridine and tertiary amines, such as triethylamine. In case a catalyst is used, it is preferred to use dichloromethane as solvent or to use no solvent at all.

The Applicant has also surprisingly noticed that, under solvent conditions, certain basic additives such as pyridine and tertiary amines, such as triethylamine, stabilize the reactants which allows an excess of the halofuran of formula (II) to be used in order to increase the conversion of the Diels-Alder reaction. The excess of the halo furan used may be recovered once the reaction is completed.

The reaction may be carried out at any suitable temperature, for example from about −80 to about 120° C., preferably from about −20 to about 100° C., more preferably from about −10 to about 80° C., for a time sufficient to convert the starting compounds into the desired Diels-Alder adduct, such as about 10 minutes to about 6 days, preferably about 3 hours to about 4 days, more preferably about 3 hours to about 2 days. The reaction can be carried out at ambient pressure or under increased pressure. In a preferred embodiment, the reaction is carried out at ambient pressure, such as about 1 bar, or at a pressure of up to 10 bars, preferably up to 5 bars, more preferably up to 2 bars.

According to an embodiment of the invention, the molar ratio of the halofuran of formula (II) to vinylhydrocarbylketone of formula (III) is close to stoichiometry (namely 1/1), or above.

The Applicant has found that a molar ratio of the halofuran of formula (II) to the vinylhydrocarbylketone of formula (III) greater than 1/1 allows for the displacement of the Diels-Alder reaction towards the formation of the cycloadduct of formula (IV).

The process according to the invention may comprise a further step of isolation of compound of formula (IV). Suitable methods of isolation and purification are known to the person skilled in the art. For example, chromatography techniques, such as liquid chromatography, may be particularly used in the process according to the invention.

Alternatively, the mixture obtained at the end of the Diels-Alder reaction and comprising structural isomers and eventually byproducts can be directly used in the subsequent step of dehydration/aromatization explained below.

In a subsequent step (step b) according to the invention, a dehydration/aromatization reaction is conducted on the compound of formula (IV) in order to provide a (para-halogeno)phenyl hydrocarbyl ketone of formula (V).

The reaction conditions for aromatization of the compound of formula (IV) are known to a person skilled in the art.

According to an embodiment of the invention, the aromatization reaction of the compound of formula (IV) requires basic reaction conditions, for example in the presence of an alkoxide compound or hydroxide compound. Suitable alkoxides may be selected from alkali methoxides, such as sodium methoxide and potassium methoxide, preferably sodium methoxide. Suitable hydroxides may be selected from alkali hydroxides, such as sodium hydroxide, potassium hydroxide, lithium hydroxide, cesium hydroxide, preferably sodium hydroxide and potassium hydroxide, more preferably sodium hydroxide.

Suitable solvents for the aromatization reaction may be for example a sulfoxide such as DMSO or an alcohol such as isopropanol.

The aromatization reaction may be carried out at any suitable temperature, for example from about −10 to about 120° C., preferably from about −5 to about 80° C., more preferably from about 0 to about 40° C.

As another subsequent step (step c) according to the invention, the compound of formula (V) is alpha-methylenated in order to provide a (para-halogeno) phenyl ketone of formula (VI):

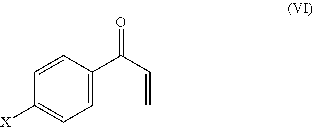

Alpha-methylenation is a known reaction for the one skilled in the art. For example, it may be implemented by heating (60 to 80° C.) the compound (V) in an aprotic solvent like THF or MeTHF in the presence of a formaldehyde source (paraformaldehyde, trioxane . . . ) and a catalyst (an acid salt of a secondary amine) (see Chem. Comm., 2010, 46, pp. 1715-1717).

In the case the radical R of the compound of formula (V) is ethyl, an alternative to the alpha-methylenation reaction is to perform a dehydrogenation reaction on said compound which will lead to the same product of formula (VI).

Once the compound of formula (VI) has been obtained, a second Diels-Alder reaction is conducted either directly on the reaction products prepared in the previous step and containing the compound (VI), or on the sole compound (VI) after separation thereof from the reaction products.

The conditions to implement said reaction may be, and preferably are, the same as, or similar to, the ones that were used in the first Diels-Alder reaction (step a1) detailed above. Most importantly, the use of the same halofuran of formula (II) must be respected.

The implementation of said second Diels-Alder reaction will lead to the formation of a second cycloadduct of formula (VII):

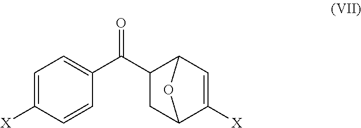

As already mentioned, compounds of formula (VII) are new and useful products per se, and are therefore also part of the present invention.

The present invention therefore also provides a process for the production of compound of formula (VII), comprising reacting a halofuran of formula (II) with a (para-halo)phenyl vinyl ketone of formula (VI), said two formulae being the same as defined before, and optionally further isolating compound of formula (VII).

Again, it has been surprisingly found that a Diels-Alder reaction efficiently occurs between the halo furan of formula (II) and the (para-halo)phenyl vinyl ketone of formula (VI) resulting in the formation of a new cycloadduct of formula (VII) as defined above. Moreover, it has been surprisingly found that said Diels-Alder reaction leads to the formation of compounds of formula (VII) with a good conversion, a low amount of by-products and a good selectivity in the desired product.

The Diels-Alder reaction between the halofuran of formula (II) and the (para-halo)phenyl vinyl ketone of formula (VI) leads to the formation of two main structural isomers represented below, one of which being the compound of formula (VII):

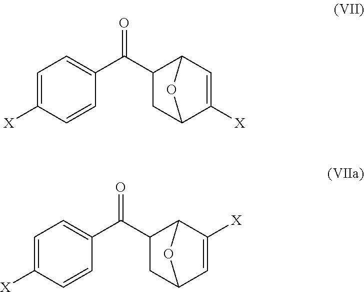

According to an embodiment of the invention, the molar ratio of compounds of formula (VII) to the total of compounds of formulae (VIIa) is of at least 50/50, preferably at least 70/30, more preferably at least 80/20.

According to an embodiment of the invention, the conversion of the second Diels-Alder reaction is of at least 50%, preferably at least 70%, more preferably at least 90%.

According to an embodiment of the invention, the molar ratio of the halofuran of formula (II) to (para-halo)phenyl vinyl ketone of formula (VI) is close to stoichiometry (namely 1/1), or above.

Like for the process for the preparation of the cycloadduct compound of formula (IV), the process according to the invention may comprise a further step of isolation of compound of formula (VII). Suitable methods of isolation and purification are known to the person skilled in the art. For example, chromatography techniques, such as liquid chromatography, may be particularly efficient in the process according to the invention.

At last, owing to a final dehydration/aromatization reaction conducted on said compound of formula (VII) (step e), the desired 4,4′-dihalobenzophenone will be ultimately obtained.

The conditions to implement said final dehydration/aromatization reaction may be, and preferably are, the same as, or similar to, the ones that were used in the first dehydration/aromatization reaction (step b) already detailed above.

The present invention also relates to a process for the direct preparation of a 4,4′-dihalobenzophenone of formula (I):

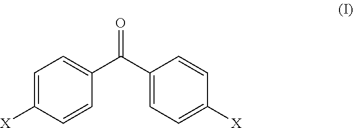

wherein X represents a halogen atom selected from the group consisting of fluorine, chlorine, bromine and iodine, comprising carrying out the dehydration/aromatization of the compound of formula (VII) as defined above.
Preferably, X is selected from fluorine and bromine, more preferably fluorine.

Another object of the invention relates to a process for producing a 4,4′-dihalobenzophenone of formula (I):

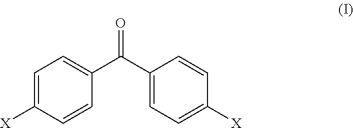

wherein X represents a halogen atom selected from the group consisting of fluorine, chlorine, bromine and iodine,
comprising:
a) providing a compound of formula (VII) as defined above,
b) carrying out the dehydration/aromatization of said compound of formula (VII).

Alternatively, 4,4′-difluorobenzophenone may be prepared by effecting a halogen-fluorine exchanging reaction between compound of formula (I) wherein X is chlorine, bromine or iodine, and an alkali fluoride, such as potassium fluoride, as described in U.S. Pat. No. 4,453,009.

According to an embodiment of the invention, 4,4′-dihalobenzophenone of formula (I) is further isolated by separation methods known to the person skilled in the art. For example, recrystallization may be carried out according to the method described in document U.S. Pat. No. 4,873,372.

Poly(aryl ether ketone)s (PAEK) are a well known class of engineering polymers useful in various fields of scientific and commercial endeavour. PAEK polymers are generally prepared by aromatic nucleophilic substitution of both the halogenides of a 4,4′-dihalobenzophenone (especially, by aromatic nucleophilic substitution of both the fluorides of 4,4′-difluorobenzophenone) by a nucleophilic oxygen atom from a co-monomer. For example, p-hydroquinone, commonly referred to as “hydroquinone” and/or at least one bisphenol (such as bisphenol A, bisphenol S, bisphenol O, 4,4′-dihydroxybiphenyl and mixtures thereof) can be used as the co-monomer; the hydrogen atom from each of the two aromatic hydroxyl groups of p-hydroquinone and/or of the bisphenol is advantageously deprotonated with at least one base (such as NaOH, Na₂CO₃, K₂CO₃ and mixtures thereof) to form a nucleophile which reacts with the 4,4′-dihalobenzophenone (preferably, 4,4′-difluorobenzophenone) to form a PAEK polymer via a nucleophilic substitution mechanism, with the halogen atoms of the 4,4′-dihalobenzophenone acting as leaving groups. Processes for preparing PAEK polymers, including those using a 4,4′-dihalobenzophenone such as 4,4′-difluorobenzophenone, can be found notably in U.S. Pat. Nos. 3,953,400, 3,956,240, 3,928,295, and 4,176,222, all incorporated herein by reference.

Then, facets of the present invention relate to:
the use of a 4,4′-dihalobenzophenone of formula (I) prepared from a compound of formula (VII) as previously defined for the manufacture of a poly(aryl ether ketone), in particular poly(ether ether ketone),
the use of a compound of formula (VII) as previously defined for the manufacture of a poly(aryl ether ketone), in particular poly(ether ether ketone), typically with a 4,4′-dihalobenzophenone of formula (I) as synthesis intermediate,
the use of a 4,4′-dihalobenzophenone of formula (I) as synthesis intermediate in the manufacture of a poly(aryl ether ketone) [in particular poly(ether ether ketone] from a compound of formula (VII) as previously defined,
the use of a 4,4′-dihalobenzophenone of formula (I) prepared from a compound of formula (IV) as previously defined wherein R is a methyl radical for the manufacture of a poly(aryl ether ketone), in particular poly(ether ether ketone),
the use of a compound of formula (IV) as previously defined wherein R is a methyl radical for the manufacture of a poly(aryl ether ketone), in particular poly(ether ether ketone), typically with a 4,4′-dihalobenzophenone of formula (I) as synthesis intermediate, and
the use of a 4,4′-dihalobenzophenone of formula (I) as synthesis intermediate in the manufacture of a poly(aryl ether ketone) [in particular poly(ether ether ketone] from a compound of formula (IV) as previously defined wherein R is a methyl radical.

Precisely, an object of the present invention concerns a process for the manufacture of a (poly aryl ether ketone), said process comprising the following steps:

i) providing a compound of formula (VII) as defined above,
ii) carrying out the dehydration/aromatization of said compound of formula (VII) to prepare a 4,4′-dihalobenzophenone of formula (I) as defined above,
iii) optionally, isolating the 4,4′-dihalobenzophenone,
iv) optionally, effecting a halogen-fluorine exchanging reaction between the 4,4′-dihalobenzophenone and an alkali fluoride as explained above,
v) carrying out the polycondensation of the 4,4′-dihalobenzophenone with at least one aromatic compound comprising 2 hydroxyl groups, in particular with hydroquinone and/or at least one bisphenol, preferably in the presence of at least one base.

Should the disclosure of any patents, patent applications, and publications which are incorporated herein by reference conflict with the description of the present application to the extent that it may render a term unclear, the present description shall take precedence.

The present invention will now be illustrated by the following examples, which are not intended to be limiting.

### EXAMPLES

### Example 1: Preparation of 4,4′-dibromobenzophenone (compound of formula (I)) from 4′-bromoacetophenone (compound of formula (V))

### 1/ Preparation of 4′-Bromoacetophenone

In a carousel tube fitted with a PTFE septum screw cap methylvinylketone (190 mg, 2.7 mmol) was weighted. Then, 3-bromofuran (2.0 g, 13.6 mmol) and diisopropylethylamine (50 mg) were added. The reaction mixture was stirred at 60° C. during 48 h. ¹H NMR analysis shows a 87% conversion of methylvinylketone to Diels-Alder adducts. For example, one of the isomers of Diels-Alder adducts have the following NMR signature: ¹H NMR (400 MHz, DMSO-d6) δ: 6.55 (d, J=2.0 Hz, 1H), 5.15 (m, 1H), 4.85 (d, J=4.8 Hz, 1H), 2.68 (dd, J=8.4, 4.0 Hz, 1H), 2.19 (s, 3H), 1.98 (dt J=11.2, 4.4 Hz, 1H), 1.52 (dd, J=11.6, 8.4 Hz, 1H).

DMSO (0.75 mL) was added to the reaction media and the mixture was transferred at 20° C. in a Schlenk tube under argon atmosphere containing powdered sodium hydroxide (47 mg, 1.18 mmol). The reaction mixture was stirred at 40° C. during 23 h. GC titration of the final reaction mixture showed the formation of 175 mg of 4′-bromoacetophenone (37% yield).

### 2/ Alpha-Methylenation of 4′-Bromoacetophenone

In a 250 ml triple-neck round-bottom flask with condenser and magnetic stirring bar, 4′-bromoacetophenone (5.0 g; 25.1 mmol) was weighted. Then, MeTHF (62.8 mL) and diisopropylamine (5.2 mL, 37.6 mmol) were added. The mixture was cooled to 10° C. and trifluoroacetic acid (1.9 mL, 25.1 mmol) was added in 5 minutes. Then paraformaldehyde (2.26 g, 75.4 mmol) was added and the mixture was heated at 70° C. during 16 h.

After cooling, water (50 mL) was added and the product is extracted with dichloromethane (3×50 mL). The combined organic layers were dried over MgSO₄ and concentrated in vacuo to afford 6.9 g of crude product.

This product was purified by silica gel chromatography (cyclohexane/ethyl acetate) to afford 3.1 g of 4′-bromoacrylophenone (compound of formula (VI)).

### 3/ Diels-Alder Reaction with 3-Bromofuran

In a carousel tube fitted with a PTFE septum screw cap 4′-bromoacrylophenone (462 mg, 2.2 mmol) was weighted. Then, 3-bromofuran (2.1 g, 14.2 mmol) and diisopropylethylamine (50 mg) were added. The reaction mixture was stirred at room temperature during 27 h. ¹H NMR show a 97% conversion of 4′-bromoacrylophenone to Diels-Alder adducts.

### 4/Aromatization of the Diels-Alder Adducts

DMSO (1.5 mL) was added to the crude reaction media from the previous example and the mixture was transferred in a Schlenk tube under argon atmosphere containing powdered sodium hydroxide (55 mg, 1.4 mmol). The reaction mixture was stirred at 40° C. during 3 h. GC titration of the final reaction mixture showed the formation of 127 mg of 4,4′-dibromobenzophenone and 10 mg of 3,4′-dibromobenzophenone, corresponding to a yield of 19% in dibromobenzophenones and a 4,4′/3,4′ ratio of 93/7.

### Example 2: Influence of a Solvent on the Diels-Alder Reaction (Step a1)

Protocol:

In a carousel tube fitted with a PTFE septum screw cap ethylvinylketone (143 mg, 1.7 mmol) was weighted. Then, 3-bromofuran (500 mg, 3.4 mmol) and the indicated solvent (0.5 mL) were added. The reaction mixture was stirred at 60° C. during 20 h. No catalyst is used. The conversion and the selectivity to Diels-Alder adducts are analyzed by NMR.

NMR (DMSO-d6, δ ¹H [δ ¹³C]):

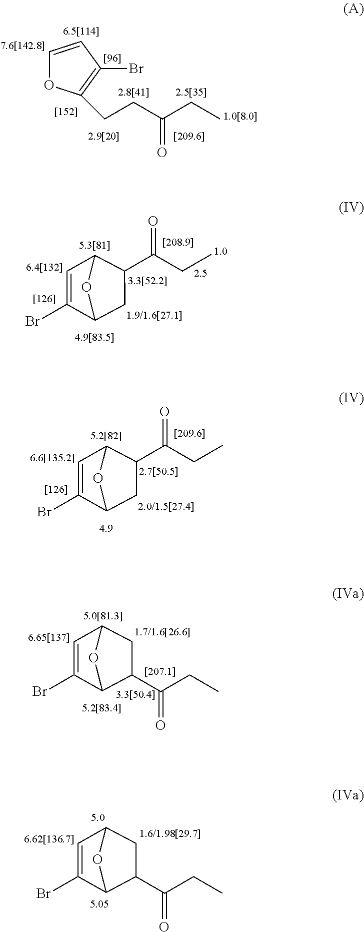

The para adducts of formula (IV) are present as exo or endo isomers. The same applies to the meta adducts of formula (IVa).

Results:

TABLE 1

| | | Molar ratio |
|---|---|---|
| Solvent | TR (%) | X/A |
| no solvent added | 94 | 47/43 |
| Diethylether (50 wt %) | 84 | 85/15 |
| DCM (50 wt %) | 85 | 75/25 |
| Pyridine (50 wt %) | 65 | 100/0 |
| DIPEA (50 wt %) | 56 | 100/0 |
| DIPEA (1 drop) | 80 | 100/0 |

TR: transformation rate

X: total amount of the four isomers (IV) and (IVa)

DCM: dichloromethane

DIPEA: diisopropylethylamine

### Example 3: Influence of a Catalyst on the Diels-Alder Reaction (Step a1)

Protocol:

In a carousel tube fitted with a PTFE septum screw cap the catalyst (0.5 mmol) was weighted and dichloromethane (3.5 mL) was added. The reaction mixture was cooled to the indicated temperature and the ethylvinylketone (286 mg, 3.4 mmol) and 3-bromofuran (500 mg, 3.4 mmol) were added. The reaction mixture was stirred at the indicated temperature during 5 h.

Then a saturated aqueous solution of sodium carbonate (2.5 mL) was then added and the mixture was allowed to warm to room temperature.

The conversion and the selectivity to Diels-Alder adducts were estimated by NMR analysis of the organic phase.

The composition of the reaction products is given in table 2.

TABLE 2

| | | Reactants | | | | Regioselectivity of the | | | |
|---|---|---|---|---|---|---|---|---|---|
| | | (mol %) | | Products | | Diels-Alder adducts IV | | | |
| | | 3- | ethylvinyl | (mol %) | | (mol %) | | | |
| | | bromofuran | ketone | X | A | meta (IVa) | | para (IV) | |
| AlCl₃ | −40° C., | 52 | 0 | 39 | 9 | 13 | 36 | 33 | 18 |
| | 15 min | | | | | | | | |
| AlCl₃ | −78° C., 5 h | 14 | 6 | 80 | 0 | 5 | 1 | 59 | 35 |
| HfCl₄ | −78° C., 5 h | 8 | 2 | 88 | 2 | 4 | 5 | 65 | 26 |
| BF₃Et₂O | −78° C., 5 h | 17 | 4 | 69 | 10 | low resolution NMR | | | |
| MgI₂ | 25° C., 1 h, | 57 | 2 | 41 | 0 | 9 | 6 | 68 | 17 |
| | neat, 3 eq | | | | | | | | |
| | bromofuran | | | | | | | | |

X: total amount of the four isomers (IV) and (IVa)
The four left columns give the propotion (mol %) of the 4 isomers of Diels-Alder adduct (IV and IVa can be endo or exo)
A: amount of product A

1.-3. (canceled)

4. A compound of formula (IV): 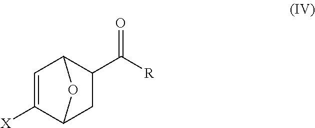
wherein X represents a halogen atom selected from the group consisting of fluorine, chlorine, bromine and iodine, and R is a hydrocarbyl radical.

5. The compound according to claim 4, wherein the halogen atom is selected from bromine and chlorine.

6. The compound according to claim 4, wherein R is a methyl radical.

7. A process for the preparation of a compound of formula (IV) as defined in claim 4, the process comprising reacting a halofuran of formula (II) with a vinylhydrocarbylketone of formula (III): 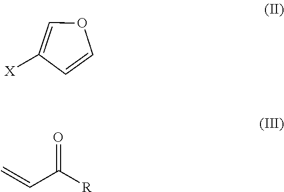
in which X represents a halogen atom selected from the group consisting of fluorine, chlorine, bromine and iodine, and R is a hydrocarbyl radical, and optionally further isolating compound of formula (IV).

8. The process according to claim 7, wherein the molar ratio of the halofuran of formula (II) to the vinylhydrocarbylketone of formula (III) is of at least 1/1.

9. A compound of formula (VII): 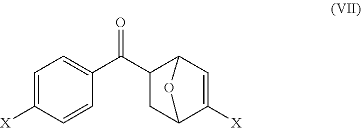
wherein X represents a halogen atom selected from the group consisting of fluorine, chlorine, bromine and iodine.

10. The compound according to claim 9, wherein the halogen atom is selected from bromine and chlorine.

11. A process for the preparation of a compound of formula (VII) as defined in claim 9, comprising reacting a halofuran of formula (II): 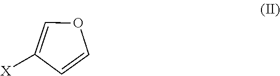
with a (p-halogeno)phenyl vinyl ketone of formula (VI): 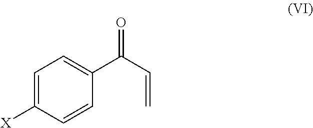
wherein X represents a halogen atom selected from the group consisting of fluorine, chlorine, bromine and iodine, and optionally further isolating compound of formula (VII).

12. The process according to claim 11, wherein the molar ratio of the halofuran of formula (II) to the (p-halogeno)phenyl vinyl ketone of formula (VI) is of at least 1/1.

13. A process for the preparation of a 4,4′-dihalobenzophenone of formula (I): 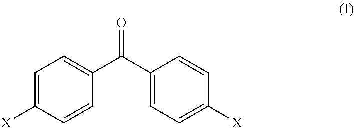
wherein X represents a halogen atom selected from the group consisting of fluorine, chlorine, bromine and iodine,
comprising carrying out the dehydration/aromatization of a compound of formula (VII): 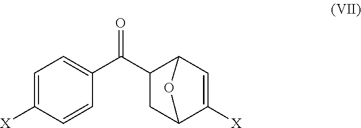
wherein X is as defined above.

14. The process according to claim 13 for the preparation of a 4,4′-dihalobenzophenone of formula (I): 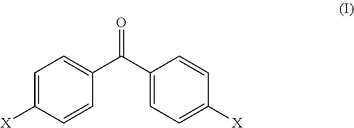
wherein X represents a halogen atom selected from the group consisting of fluorine, chlorine, bromine and iodine,
comprising:
a) providing a compound of formula (VII): 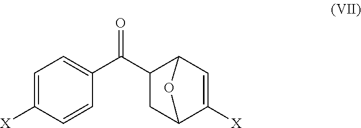
wherein X is as defined above,
b) carrying out the dehydration/aromatization of said compound of formula (VII),
c) optionally further isolating compound of formula (I).

15. (canceled)

16. The process according to claim 14 for the preparation of a 4,4′-dihalobenzophenone of formula (I): 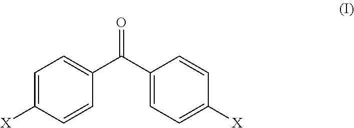
wherein X represents a halogen atom selected from the group consisting of fluorine, chlorine, bromine and iodine,
said process comprising the steps of:
a1) reacting a halofuran of formula (II) with a vinylhydrocarbylketone of formula (III): 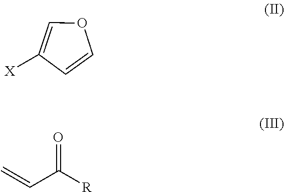
wherein X is the same as defined above, and R is a methyl radical,
thereby providing reaction products containing a cycloadduct compound of formula (IV): 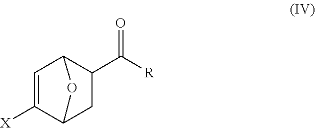
in which X and R are the same as defined above;
a2) optionally further isolating said compound of formula (IV);
b) carrying out a dehydration/aromatization of said adduct compound of formula (IV), thereby providing reaction products containing a (p-halogeno)phenyl hydrocarbyl ketone of formula (V): 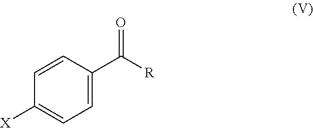
in which X and R are the same as defined above;
c) carrying out an alpha-methylenation of said compound of formula (V), thereby providing reaction products containing a (p-halogeno)phenyl vinyl ketone of formula (VI): 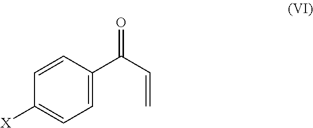
in which X is the same as defined above;
d1) reacting said compound of formula (VI) with the same halofuran of formula (II) as defined above, thereby providing reaction products containing a cycloadduct compound of formula (VII): 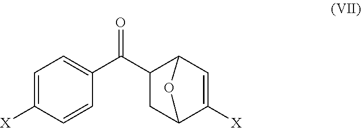
in which X is the same as defined above;
d2) optionally further isolating said compound of formula (VII);
e) carrying out a dehydration/aromatization of said adduct compound of formula (VII), thereby providing reaction products containing said 4,4′-dihalobenzophenone of formula (I).

17. The process according to claim 16, wherein the dehydration/aromatization is carried out in the presence of an alkali methoxide or an alkali hydroxide.

18. The process according to claim 17, wherein the alkali methoxide is sodium methoxide or potassium methoxide.

19. The process according to claim 17, wherein the alkali hydroxide is sodium hydroxide or potassium hydroxide.

20. The process according to claim 16, wherein the dehydration/aromatization is carried out in the presence of DMSO or an alcohol.

21. A process for the manufacture of a (poly aryl ether ketone), said process comprising the following steps:
i) providing a compound of formula (VII) according to claim 9,
ii) carrying out the dehydration/aromatization of said compound of formula (VII) to prepare a 4,4′-dihalobenzophenone of formula (I): 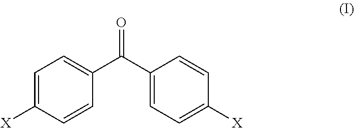
wherein X represents a halogen atom selected from the group consisting of fluorine, chlorine, bromine and iodine,
iii) optionally, isolating the 4,4′-dihalobenzophenone,
iv) optionally, effecting a halogen-fluorine exchanging reaction between the 4,4′-dihalobenzophenone and an alkali fluoride,
v) carrying out the polycondensation of the 4,4′-dihalobenzophenone with at least one aromatic compound comprising 2 hydroxyl groups, optionally in the presence of at least one base.

22. The process according to claim 21, wherein the at least one aromatic compound comprising 2 hydroxyl groups is hydroquinone.

23. The compound according to claim 5, wherein the halogen atom is chlorine.

24. The compound according to claim 10, wherein the halogen atom is chlorine.
